(19) United States
(12) Patent Application Publication
(10) Pub. No.: US 2022/0143081 A1
(43) Pub. Date: May 12, 2022

(54) SYSTEM AND METHOD OF TREATING INFLAMMATORY LUNG DISEASES

(71) Applicant: Otto M. Ah Ching, JR., Greer, SC (US)

(72) Inventor: Otto M. Ah Ching, JR., Greer, SC (US)

(21) Appl. No.: 17/523,105

(22) Filed: Nov. 10, 2021

Related U.S. Application Data
(60) Provisional application No. 63/112,059, filed on Nov. 10, 2020.

(51) Int. Cl. A61K 33/40; A61K 9/08; A61K 9/00

(52) U.S. Cl. CPC A61K 33/40; A61K 9/0043; A61K 9/08

(57) ABSTRACT

TCDO can be composed of stabilized chlorite ions that target white blood cells to balance the immune system. TCDO may be applied nasally or via inhaler directly to the nasal passages or the lungs to treat lung diseases. TCDO can be provided in 20-mL vials as a sterile, clear, colorless, odorless, isotonic aqueous solution for intravenous, nasal, or lung administration.

### CROSS REFERENCE TO RELATED APPLICATIONS

The present application claims the benefit under 35 U.S.C. 119 of U.S. Provisional Patent Application Ser. No. 63/112,059 filed Nov. 10, 2020. The U.S. Provisional Patent Application Ser. No. 63/112,059 is hereby incorporated by reference in its entirety.

### BACKGROUND OF THE INVENTION

### Field of the Invention

The present specification relates to a system and method of treating inflammatory lung diseases and even more particularly for treating inflammation in a user due to COVID-19.

### Description of Related Art

Due to the rapid onslaught attack of COVID-19, scientists are rushing to seek anti-viral therapies and vaccines in hopes of mitigating the deleterious effects and mortality associated with COVID-19. The COVID-19 virus like other similar Corona viruses produces certain inflammatory cytokines and chemokines mediated by primary monocyte or macrophage regulation and modulation. When the primary system is overwhelmed, an alternative activated macrophage pathway (“AAMP”) becomes enhanced and proliferation of inflammatory cytokines are illicitly released and provoke irregular T-cell responses leading to decompensating and detrimental insult to tissue and organ damage.

Other agents being studied include the IL-6 receptor blocker tocilizumab (Actemra, Genentech), which the FDA has approved for the treatment of severe cytokine release syndrome in patients receiving CAR T-cell therapy. A case reported from China has shown that in some patients using the CAR T-cell therapy has successfully slowed COVID-19-associated cytokine storm (Blood Adv 2020: 14; 4[7]:1307-1310). A IL-1 blocker, anakinra (Kineret, Amgen) has been approved by the FDA to treat rheumatoid arthritis and an infantile multisystem inflammatory disease, and emapalumab (Gamifant, Novimmune/Sobi) for hemophagocytic lymphohistiocytosis, the genetic predisposition to cytokine storm.

Glucocorticoids influence all types of inflammatory events, no matter their cause. Glucocorticoids induce the lipocortin-1 (annexin-1) synthesis, which then binds to cell membranes preventing the phospholipase A2 from coming into contact with its substrate arachidonic acid, which can lead to diminished eicosanoid production. The cyclooxygenase (both COX-1 and COX-2) expression is also suppressed, potentiating the effect. Glucocorticoids can also stimulate the lipocortin-1 escaping to the extracellular space where it binds to the leukocyte membrane receptors and inhibits various inflammatory events such as epithelial adhesion, emigration, chemotaxis, phagocytosis, respiratory burst, and the release of various inflammatory mediators (lysosomal enzymes, cytokines, tissue plasminogen activator, chemokines, etc.) from neutrophils, macrophages, and mastocytes.

The symptoms that are most commonly reported include cough, fever, and shortness of breath. The pathophysiology of the disease explains why respiratory symptoms are so common: indeed, the virus accesses host cells via the protein angiotensin-converting enzyme (ACE2) which is very abundant in the lungs. Nevertheless, ACE2 is also expressed by endothelial cells (ECs), and other major clinical events usually observed in COVID-19 patients (e.g. high blood pressure, thrombosis, pulmonary embolism) seem to suggest that the virus is targeting the endothelium, one of the largest organs in the human body.

Infectious diseases, neurodegenerative diseases, autoimmune diseases, and effective wound care repair are facing unmet therapeutic needs that are the primary causes of morbidity and mortality worldwide. Despite many years of research and development, changes in patient mortality have only improved slightly. Across the traditional standard of care therapy segments including chemotherapy, steroids, antiviral and immune modulators, unsatisfied patients continue to demand new and improved solutions meeting a wide spectrum of needs. These needs involve costs, efficacy, safety, utility, duration and improved outcome.

Referring initially to FIG. 1, the body's response and injuries associated with COVID-19 from acute insult to chronic multi-organ damage via the respiratory tract and how diagnosis is made via symptomatic and PCR testing to confirm findings is shown. However, almost all the emphasis is placed on how the virus attacks the body and on preventing infection and the body's own response is completely ignored. PCR swab test kits have proven the presence of antibodies specifically IgG and IgM, however it would be advantageous to also consider certain pro-inflammatory cytokines which could benefit the different stages of COVID-19 infection and its lingering effects. Cytokine panels currently exist and could be added as a another process the diagnosis of the disease. For this reason, it would be appropriate to test early and utilize a potential prophylactic that will assist in mitigating the harmful effects and progression of the disease process. Therefore, there is a need for a product that targets infectious diseases, neurodegenerative diseases, autoimmune diseases, and effective wound care repair and meets a wide spectrum of needs that offer efficacy, safety, utility, duration and improved outcome.

### BRIEF SUMMARY OF THE INVENTION

The objectives of the invention may be achieved through a system providing a therapeutic amount of tetrachlorodecaoxide (TCDO) is provided to a patient. The TCDO may be provided in a solution in a concentration of about 20% by volume to about 30% by volume. The TCDO may be present in a concentration of 0.1 gt/mL in a saline solution. The amount of TCDO delivered to the patient may be equivalent to approximately 3.5 to approximately 5 mg of chlorite ion. The solution may also include a flavoring and/or a scent. The flavoring may be coconut oil, aloe vera, or the like and the scent may be eucalyptus and/or lavender or the like. The flavoring and scent may be present at a concentration between about 5% and 20% by volume. The solution may be stored and delivered via a nasal spray bottle to the nasal passages or via an inhaler to the lungs.

The objectives of the invention may also be achieved through methods involving treating a patient with a lung disease providing a solution comprising tetrachlorodecaoxide in a concentration of about 20% by volume to about 30% by volume and delivering between about 0.5 mL to 1.0 ML of the solution to the patient. The lung disease may be COVID-19. In some embodiments, the tetrachlorodecaoxide is diluted to approximately 250 mL or more of 0.9% sodium chloride solution. The solution may be stored and delivered via a nasal spray bottle to the nasal passages or via an inhaler to the lungs. The solution may be aerosolized immediately before delivery of the solution to the patient.

A dose of the solution may be provided to the patient twice daily for between three and five days. The dose may be between 0.5 mL of 20% tetrachlorodecaoxide by volume and 1 mL of 30% tetrachlorodecaoxide. The solution may be directly delivered to the nasal cavity or the rhinopharynx via a nasal spray bottle, or the lungs of the patient via an inhaler.

Aspects and applications of the invention presented here are described below in the drawings and detailed description of the invention. Unless specifically noted, it is intended that the words and phrases in the specification and the claims be given their plain, ordinary, and accustomed meaning to those of ordinary skill in the applicable arts. The inventors are fully aware that they can be their own lexicographers if desired. The inventors expressly elect, as their own lexicographers, to use only the plain and ordinary meaning of terms in the specification and claims unless they clearly state otherwise and then further, expressly set forth the. Absent such clear statements of intent to apply a “special” definition, it is the inventor's intent and desire that the simple, plain, and ordinary meaning to the terms be applied to the interpretation of the specification and claims.

The inventors are also aware of the normal precepts of English grammar. Thus, if a noun, term, or phrase is intended to be further characterized, specified, or narrowed in some way, then such noun, term, or phrase will expressly include additional adjectives, descriptive terms, or other modifiers in accordance with the normal precepts of English grammar. Absent the use of such adjectives, descriptive terms, or modifiers, it is the intent that such nouns, terms, or phrases be given their plain, and ordinary English meaning to those skilled in the applicable arts as set forth above.

Further, the inventors are fully informed of the standards and application of the special provisions of 35 U.S.C. § 112 (f). Thus, the use of the words “function,” “means” or “step” in the Detailed Description or Description of the Drawings or claims is not intended to somehow indicate a desire to invoke the special provisions of 35 U.S.C. § 112 (f), to define the invention. To the contrary, if the provisions of 35 U.S.C. § 112 (f) are sought to be invoked to define the inventions, the claims will specifically and expressly state the exact phrases “means for” or “step for, and will also recite the word “function” (i.e., will state “means for performing the function of [insert function]”), without also reciting in such phrases any structure, material or act in support of the function. Thus, even when the claims recite a “means for performing the function of . . . ” or “step for performing the function of . . . ,” if the claims also recite any structure, material or acts in support of that means or step, or that perform the recited function, then it is the clear intention of the inventors not to invoke the provisions of 35 U.S.C. § 112 (f). Moreover, even if the provisions of 35 U.S.C. § 112 (f) are invoked to define the claimed inventions, it is intended that the inventions not be limited only to the specific structure, material or acts that are described in the preferred embodiments, but in addition, include any and all structures, materials or acts that perform the claimed function as described in alternative embodiments or forms of the invention, or that are well known present or later-developed, equivalent structures, material or acts for performing the claimed function.

Additional features and advantages of the present specification will become apparent to those skilled in the art upon consideration of the following detailed description of the illustrative embodiment exemplifying the best mode of carrying out the invention as presently perceived.

### BRIEF DESCRIPTION OF THE DRAWINGS

These and other features, aspects, and advantages of the present specification will become better understood with regard to the following description, appended claims, and accompanying drawings where:

FIG. 1 is an illustration depicting the body's response and injuries associated with COVID-19 in accordance with the prior art;

FIG. 2 is a graphic depiction of the uncoupling of hemoglobin between team compound and ferritin and porphyrin in accordance to one, or more embodiments;

FIG. 3 is a graph depicting the response of TCDO with HIV-AIDS patients with comorbidities or opportunistic infections in accordance to one, or more embodiments;

FIG. 4 is a graphic depicting activity of TCDO in accordance to one, or more embodiments;

FIG. 5 is an illustration of a macrophage targeted therapeutic requires an understanding of the role of macrophages in human disease in accordance to one, or more embodiments;

FIG. 6 is a graph depicting that TCDO may cause marked inhibition of inducible genes related to T-cell proliferation in accordance to one, or more embodiments;

FIG. 7 is a graphical depiction of the chemical structure of TCDO in accordance to one, or more embodiments;

FIG. 8 is a graphical depiction of further chemical structural components of TCDO in accordance to one, or more embodiments; and

FIG. 9 is a graph showing a reduction in TNSS over the course of the observation period in accordance to one, or more embodiments.

Elements and acts in the figures are illustrated for simplicity and have not necessarily been rendered according to any particular sequence or embodiment.

### DETAILED DESCRIPTION OF THE INVENTION

In the following description, and for the purposes of explanation, numerous specific details are set forth in order to provide a thorough understanding of the various aspects of the invention. It will be understood, however, by those skilled in the relevant arts, that the present invention may be practiced without these specific details. In other instances, known structures and devices are shown or discussed more generally in order to avoid obscuring the invention. In many cases, a description of the operation is sufficient to enable one to implement the various forms of the invention, particularly when the operation is to be implemented in software. It should be noted that there are many different and alternative configurations, devices and technologies to which the disclosed inventions may be applied. The full scope of the inventions is not limited to the examples that are described below.

In some embodiments an antimicrobial, antiviral, and antifungal agent which can be in the form of a nasal spray can help boost the user's immune system and help mitigate further detrimental effects felt by COVID-19. Referring to FIGS. 1 & 2 a separation uncoupling of hemoglobin between heme compound and ferritin and porphyrin are released into the blood stream throughout the body that can cause hemolysis and damage to the tissue and the surrounding organs which can mitigate oxygen leading to ischemia and hypoxia throughout the body. An ACE2 receptor site can bind to the virus for increased infectivity. The T cells and lymphocytes can activate the inflammatory response and release cytokines. It is during this period as with most comorbidities or secondary infections that lead to deleterious effects to the whole-body system. The infection of the virus attack is due to the primary effector and conductor of the immune response.

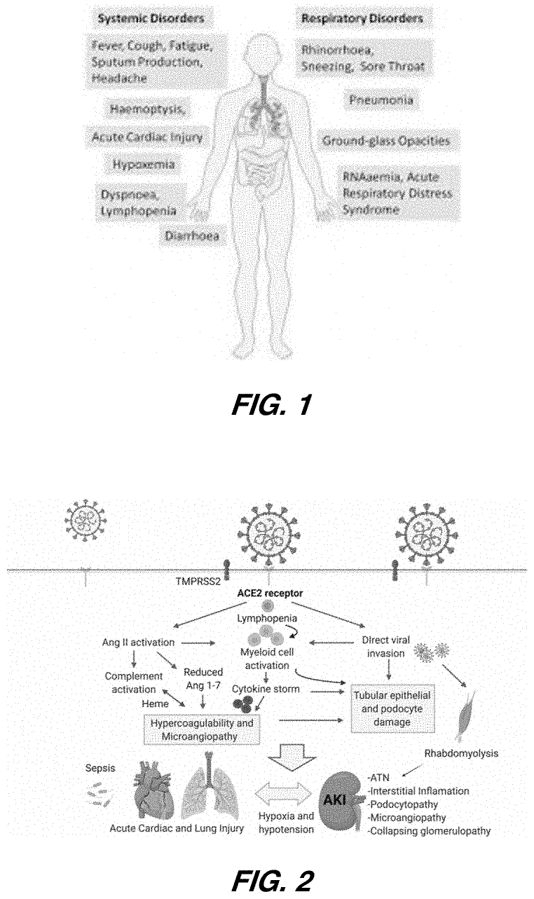

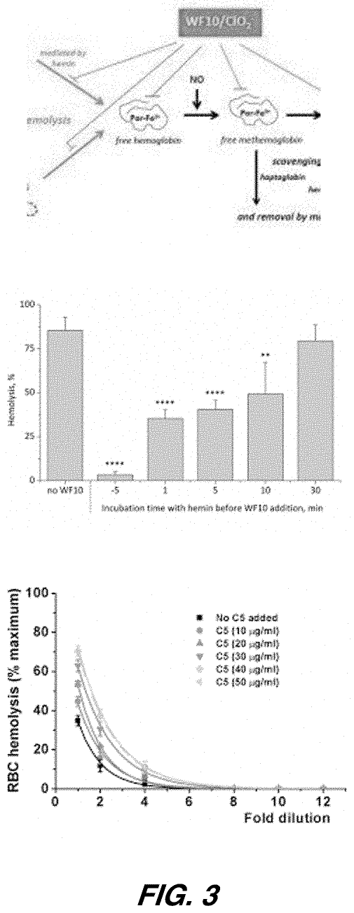

Therefore, access to the body's immune system can mitigate any deleterious threats from the virus. FIG. 2 demonstrates how the body is attacked by COVID-19 which can lead to harmful effects due to overwhelming pro-inflammatory response which leads to other multisystem damage. Similar studies have been done in HIV AIDS patients with opportunistic infections and most notable was the effect of TCDO on macrophages, but also certain T cells-CD-8 cells can assist in phagocytic activity along with macrophages that assist in blocking spike infectivity cells and can assist in clearance of the virus overtime.

A novel computational approach was applied to search among approved and clinically tested drugs from the DrugBank database. Therapeutic Candidates were selected based on entropy homology and predefined activity profiles of three small molecules with anti-SARS-CoV activity and a published data set. TCDO, an histamine 1 receptor-blocker, was predicted in multiple screens, and based on its attractive safety profile and availability in nasal formulation, was selected for experimental testing. Antiviral activity was tested in vitro in SARS-CoV-2 infection assays with Vero E6 monkey kidney epithelial cells and reconstituted human nasal tissue. The effect on viral replication was assessed by quantification of viral genomes by droplet digital PCR.

Surprisingly, TCDO significantly reduced cytopathic effect and SARS-CoV-2 infection of Vero E6 cells with an EC50 of [˜]6 M both in a preventive and treatment setting. Furthermore, TCDO in a commercially available nasal spray tested at 5-fold dilution was highly potent in inhibiting viral propagation in SARS-CoV-2 infected reconstituted human nasal tissue. Formulations of TCDO were considered as a topical prevention or treatment of nasal colonization with SARS-CoV-2. As such, TCDO was surprisingly found to be useful in reducing viral spread and prophylaxis of COVID-19.

In embodiments TCDO can help with early intervention via nasal spray into a user's respiratory system where most of these patients release pro-inflammatory IL-1, 2, 4, 6 predominately, TNF alpha.

TCDO is a stabilized chlorite solution under directive 2001/20/EC. TCDO has been approved in Thailand since 1997 for the treatment of late post-radiation cystitis caused by radiotherapy for cancer and has been studied in over 2000 patients for various indications worldwide. A topical version of OXO-K993 has been approved as a wound-healing agent under the product names Oxoferin® and Oxovasin® in Germany since 1983. The intravenous (“I.V.”) form has been approved for HIV treatments in Austria, Switzerland, Germany and Canada for compassionate use and in the USA for “right to try” patients.

The pharmacological activity of TCDO stems from its ability to regulate inappropriate immunological activation. TCDO qualitatively and quantitatively modulates the immune response by influencing both cell proliferations and function. The pharmacological effects of TCDO were quantitated in various animal models by measuring the proliferation of immunocompetent cells, phagocytosis index, oxidative burst, cell signaling, intracellular recruitment, activation of T-cells as bacteriocidal and virucidal and immunoglobulin production. TCDO stimulates phagocytosis, humoral immune response, and cellular defense systems by modifying the function of the monocyte/macrophage system, natural killer (NK) cells, and cytotoxic T-lymphocytes (CTL).

Nonclinical and clinical studies have provided evidence that TCDO down-regulates inappropriate immunologic activation through removal of the inflammatory macrophage influence by enhancing phagocytic activity on chronic T cell activation to potentially re-establish immunologic balance. Many chronic diseases are thought to arise through inappropriate immune activation, driven in part by viral (e.g., HIV, HCV) or inappropriately recognized self-antigens (i.e., autoimmune disease).

In embodiments the nasal spray can comprise TCDO as the pharmacologic main ingredient (“PMI”) for COVID-19. The amount of PMI will be used to determine the strength and duration of the treatment. Also, additional additives will be included for pleasing taste and scent in manufacturing process which can be a mixture of five to twenty percent coconut oil and five to twenty percent eucalyptus with a concentration of TCDO of at least 0.75. In a particular embodiment, a nasal spray includes 5% aloe vera and 5% lavender. The nasal spray may be provided in 25 ml nasal spray applicators at a dosage rate of 0.75 ml strength from 0.5 ml=20% normal administration rate to 0.75 ml=30% metered of 1.0 ml. twice daily for 3-4 days duration treatment time.

The nasal cavity and the rhinopharynx are key sites that harbor virus initially, replication of SARS-CoV-2. TCDO is thought to exert anti-viral activity through its interaction with the viral surface, preventing viral entry and capturing viral particles released by infected cells and increased phagocytosis activity by monocycle activity with CD-3/8 and CD-4 lymphocyte. The spray is formulated to reduce the inflammatory factors by down regulation viral load in upper respiratory airways, preventing viruses from proliferating and spreading into the lungs via alveolar macrophages. The spray is formulated to reduce the viral load in upper respiratory airways, preventing viruses from proliferating and reduce spreading into the lungs.

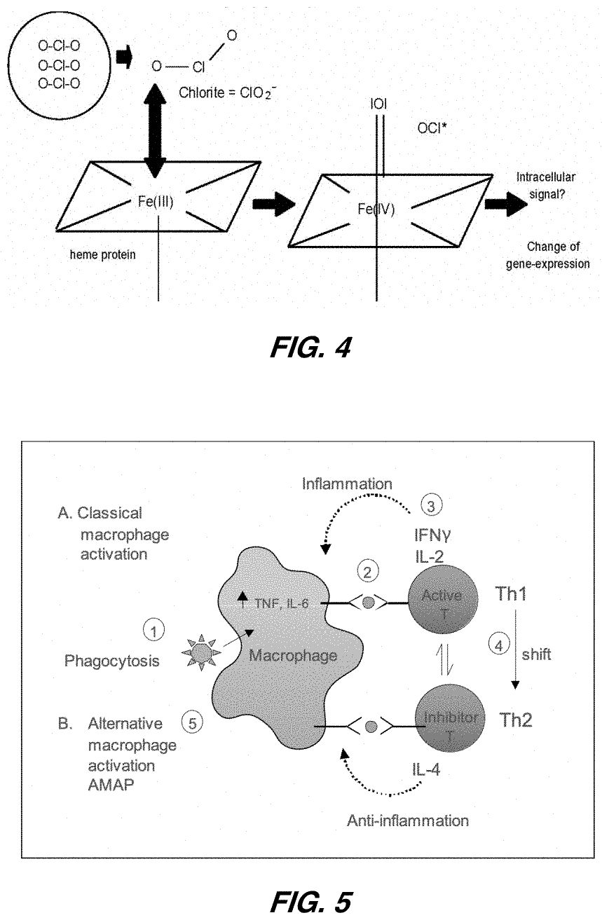

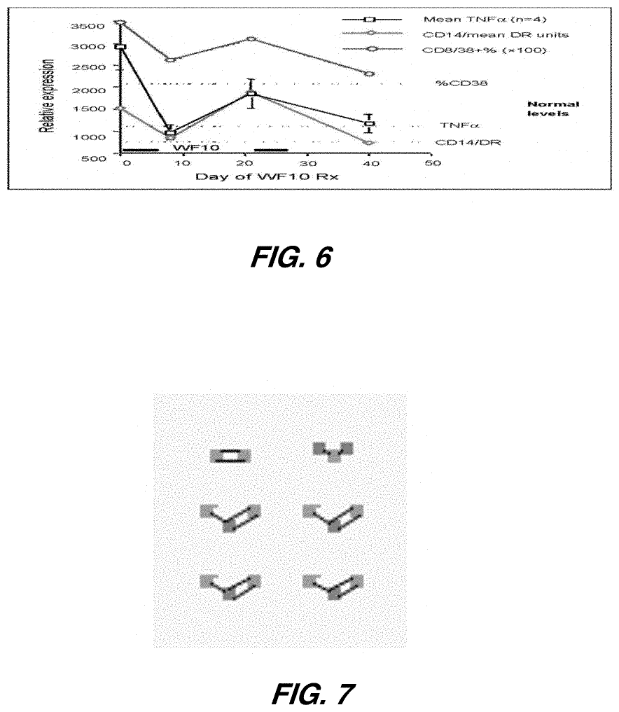

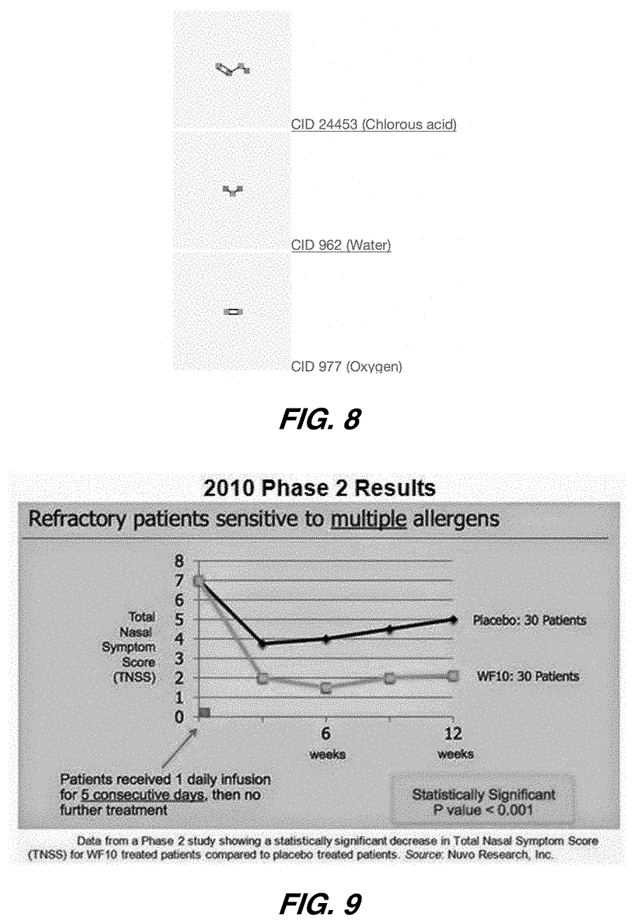

FIG. 4 shows activity of TCDO. In vitro, TCDO depends upon interaction with hemeproteins (eg, hemoglobin, myoglobin, peroxidase, cytochrome) for its activity. Upon reaction with heme protein, TCDO becomes a secondary oxidant of the compound I type, similar to a peroxide-peroxidase complex and undergoes a very rapid transformation to water, chloride ion and molecular oxygen. Chlorite released from compound 1 intermediate within macrophages is hypothesized to mediate the observed immunological and clinical effects of an antigen. The degree and speed of chloride release from TCDO depends on the nature and concentration of the hemoprotein. If peak chloride release is used as a measure of complete metabolism of TCDO, a half-life of <5 min is obtained in the presence of hemoglobin 1 μM. TCDO is undetectable within 30 s of addition of the equivalent of a 2.5 ml/kg dose to whole blood. The almost immediate breakdown of TCDO and its very short half-life, means detection of it or its metabolites (oxygen, water and chloride) in blood is not technically possible at the doses used in clinical trials (0.5 ml/kg).

The TCDO oxygen/chlorite matrix is stable until interaction with heme-associated iron, whereupon it is converted to an active chlorite (OCL*) molecule through a Michaelis-Menten reaction and intermediate production of a reactive compound. Chlorite is the active form of the drug thought to mediate the immunological effects in macrophages. A cumulative effect of the pharmacologically active species is unlikely using the recommended dosing regimen (0.5 ml/kg/day for 5 days) for I.V. administration, considering the in vivo hemoprotein concentration (2.5 mM) and the relationship between the rate of metabolism and bioactivator concentration. Biochemical studies indicate that formation of toxic oxygen species, such as singlet oxygen, hydroxyl radical, superoxide radical, or peroxide, does not occur. Formation of free chlorite or hypochlorite was not detectable.

In a series of in vitro experiments in monocytes, macrophages and lymphocytes TCDOP was observed to increase phagocytosis, stimulate an oxidative burst in monocytes, decrease macrophage tumor necrosis factor (TNF) expression, decrease antigen presentation and enhance macrophage-fibroblast cooperation. In vitro, TCDO also displayed anti-HIV properties that did not appear to be involved in the drugs macrophage targeted mechanism of action. In vivo studies investigated the effects of TCDO on monocytes, macrophages and lymphocytes, on humoral and cellular immunity, and on response to local or total body irradiation.

TCDO increased the number of macrophages infiltrating the skin blister in a human wound healing model. In rats TCDO increased the proportion of granulocytes, peripheral blood monocytes (PBMCs) and large granular lymphocytes (LGLs), and stimulated erythropoiesis after total body X-irradiation. In mice, TCDO stimulated regeneration of hematopoietic stem cells receiving sublethal doses of irradiation. In other studies, TCDO displayed direct antitumor effects against radiation-induced, chemical-induced and metastatic malignant and benign tumors. TCDO altered proportions of T-helper and T-suppressor/cytotoxic cells in spleen and thymus and increased both the humoral and cellular immune responses measured by the Jerne plaque and footpad swelling tests, respectively. TCDO (0.2 ml/kg iv) administered twice during the day before iv administration of lipopolysaccharide (LPS) from E coli 055B5 reduced the sensitivity of BALB/cABOM mice to the endotoxin, increasing the LD50 value from 346 to 518 mg. TCDO attenuation of endotoxin toxicity is consistent with the downregulation of expression of TNF observed in vitro.

Referring to FIG. 5, development of a macrophage targeted therapeutic requires an understanding of the role of macrophages in human disease. The balanced macrophage activation hypothesis proposes that the major functions of macrophages (phagocytosis, antigen presentation, and inflammation) are components of a host-regulated cycle that maintains the homeostasis of macrophage inflammatory activity. Chronic diseases are hypothesized to arise through over-representation of the inflammatory component of the cycle, driven in part by viral (eg, HIV) or inappropriately recognized self-antigens (autoimmune disease). Macrophage phagocytosis, antigen presentation and inflammation are components of a host-regulated cycle that maintains the homeostasis of macrophage inflammatory activity

Phagocytosis is the first step in macrophage activation. Macrophages engulf pathogens, such as, bacteria, fungi, and viruses. Upon successful phagocytosis of a foreign substance, the macrophage processes this material through a proteolytic pathway, cutting individual proteins into small peptides that are then involved in the second step of macrophage activation: the presentation of antigens to T-cells.

After foreign materials have been cut into peptides, macrophages present antigens to T-lymphocytes utilizing the major histocompatibility antigens class 1 (HLA) and class 2 (DR) and initiate expansion of a normal immune response (step 2). T-cell activation predominantly occurs through this antigen-presenting-cell function. Standard cytotoxic T-cells specific for virus-infected cells, cancers or fungi are developed, which ultimately leads to their successful immunological clearance. Upon successful activation, T-cells express various activation antigens, such as CD38, and secrete factors, such as IL-2, which allows T-cells to proliferate, and IFNJ, which causes further macrophage activation and step 3.

A product of T-cell activation, IFNJ induces full inflammatory changes and classical macrophage activation. This activation causes upregulation of inflammatory cytokines, such as IL-1, IL-6, and TNFD. The macrophage in this state is extremely inflammatory and causes secondary effects, such as fevers, and when chronically stimulated, weight loss and further non-specific activation of immunological responses.

During the initiation of a cellular response, which ultimately leads to production of cytotoxic T-cells and IL-2-producing Th1 cells, a second major class of T-cells, the Th2 cell, is induced. Th2 cells are involved in B-cell activation and proliferation, hypergammaglobulinemia, upregulation of IgE, eosinophilia and allergic reactions. As a net result, excess IL-2 production shuts off step 2. The Th1 and Th2 cell activation process occurs virtually simultaneously in vitro (and likely in vivo), although classical immunological responsiveness, as measured by T-cell proliferation, predominantly considers the Th1-like response.

The ability of TCDO to specifically regulate macrophage gene expression and thereby inhibit T-cell activation was examined using the recently developed Light Cycler System (Roche Molecular Biochemicals), a quantitative reverse transcriptase PCR system which allows quantitation of gene expression. In vitro studies of gene expression in PBMCs isolated from normal donors showed that TCDO causes notable inhibition of inducible gene expression related to macrophage dependent T-cell activation (IL-2, IL-4, IL-17) and causes short-term upregulation of CD14 cell (macrophage) inflammatory gene expression.

All our patients with elevated TNFD levels (>1215 copies) were analyzed for subsequent changes in TNFD gene expression using RT-PCR. Patient control specimens that had been frozen in liquid nitrogen were thawed, washed and placed in 37° C. medium for 2 hours prior to RNA extraction. TNFD gene expression was quantitated in comparison with internal housekeeping genes. HLA DR expression on CD14 expressing cells and CD38 expression on CD8 expressing cells was determined and quantitated using a FACScan. The data shown represent the average of all values obtained from the four patients over the course of this trial. The TNF⋅ gene expression parameters include standard deviation of the TNFD gene product in repeat analyses.

Referring to FIG. 7-8, the chemical structure of TCDO is shown. The nasal spray compound consists of that TCDO-60% of main ingredient diluted into 0.9% normal saline with other organic natural aromatics and flavor. One milliliter of TCDO contains approximately 0.1 g of OXO-K993/TCDO in sterile Water for Injection (Ph.Eur./USP), equivalent to approximately 4.25 mg (63 μmol) of chlorite ion (ClO2−). The molarity is calculated by the chlorite ion content: OXO-K993/TCDO and IMMUNOSTAT-IMMUNOSTATNIDEX-TCDO contain chlorite at concentrations of 693 mM and 63 mM, respectively.

One milliliter of TCDO contains approximately 0.1 g of TCDO in Water for Injection (Ph.Eur./USP), which is equivalent to approximately 4.25 mg (63 μmol) of chlorite ion (ClO2−). The molarity is calculated by the chlorite ion content: TCDO and TCDO contain chlorite at concentrations of 693 mM and 63 mM, respectively. TCDO can be provided in 20-mL vials as a sterile, clear, colorless, odorless, isotonic aqueous solution (pH of 20% solution: 10.75 to 11.9) for intravenous infusion.

Vials of TCDO must be stored in their original containers (to avoid exposure to light) at normal room temperature (not exceeding 86° F. or 30° C.). TCDO in its original, sealed bottle is stable for up to 60 months when protected from light. TCDO must be diluted within 4 hours before administration in, for example 250 mL or more of 0.9% sodium chloride injection. Refrigeration or freezer storage should not be used to extend the 4-hour limit. During dilution and administration, TCDO must be protected from direct sunlight, as well as from sunlight filtered through glass windows. The diluted TCDO in fusion solutions must be administered in a slow continuous infusion over at least 60 to 90 minutes. In some embodiments, TCDO is converted from an I.V. application to a nasal spray thus allowing the user to inhale the TCDO along with the other novel components.

In embodiments a nasal spray is used to combat COVID-19 as a prophylactic in mitigating early onset of the disease progression. The IV (intravenous) infusion form may be used for those patients who have an active progression of the disease and prevent them from acquiring further harmful damage to: lungs, liver, kidney, heart, and brain.

In embodiments, TCDO can be a nasal spray hypochlorite-hypochlorite wherein the cytokine inflammatory markers can be reduced or decreased wherein the inflammatory response can be regulated causing a cascading effect of the inflammatory cytokine wherein the focus is on more than one marker which can regulate or modulate the inflammatory response creating a balance. In embodiments the TCDO consecrates on multiple markers instead of just one marker. For TCDO to take effect the user will have to use it as a prophylactic or an IV drip two to three times daily for one to five days. The spray is a metered and can consist of five to fifteen percent coconut oil, and five to fifteen percent eucalyptus. In embodiments TCDO can be used with the following disease, Neurodegenerative Diseases, CVA Myopathy/Stroke, Wound Care, Opioid Addiction, Pain Cessation, Cystic Fibrosis, Cancer Adjunctive Therapy, Alzheimer's Disease, All Lung Diseases, RBC/WBC Expanders with No Side Effects, Antiviral Activity, Antifungal Activity, Antimicrobial Activity, and Non-Steroidal Agent.

In embodiments, TCDO can be composed of stabilized chlorite ions that target white blood cells to balance the immune system. Our novel nasal spray administration route for Covid 19 increases effectiveness of treatment by delivering the treatment directly to the site most at risk for harming the patient.

In some embodiments, TCDO is administered with a dosage of TCDO of 40-60% main ingredient (0.75) milligrams twice a day (BID) for three days. One or more additives can be added to the TCDO such as, 10% coconut oil and 10% lavender or 10% eucalyptus in our nasal spray for over-the-counter use.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only and is not intended to limit the scope of the present disclosure, which is defined solely by the claims. Accordingly, embodiments of the present disclosure are not limited to those precisely as shown and described.

Certain embodiments are described herein, including the best mode known to the inventors for carrying out the methods and devices described herein. Of course, variations on these described embodiments will become apparent to those of ordinary skill in the art upon reading the foregoing description. Accordingly, this disclosure includes all modifications and equivalents of the subject matter recited in the claims appended hereto as permitted by applicable law. Moreover, any combination of the above-described embodiments in all possible variations thereof is encompassed by the disclosure unless otherwise indicated herein or otherwise clearly contradicted by context.

What is claims is:

1. A system for treating inflammatory lung diseases, the system comprising:
a solution comprising tetrachlorodecaoxide in a concentration of about 40 w/w % to about 60 w/w %.

2. The system of claim 1, wherein the solution contains tetrachlorodecaoxide equivalent to approximately 3.5 to approximately 5 mg of chlorite ion.

3. The system of claim 1, further comprising at least one of a flavoring and a scent.

4. The system of claim 2 wherein the flavoring is at least one of coconut oil and aloe vera.

5. The system of claim 2 wherein the scent is at least one eucalyptus and lavender.

6. The system of claim 2, wherein the flavoring is coconut oil, and the scent is lavender.

7. The system of claim 2 wherein the flavoring and scent are present at a concentration between about 5 w/w % and 20 w/w %.

8. The system according to claim 1, wherein the TCDO is present at a concentration of 0.1 g/mL in a saline solution.

9. A method of a treating a patient with a lung disease, the method comprising the acts of:
providing a solution comprising tetrachlorodecaoxide in a concentration of about 20% by volume to about 30% by volume;
delivering between about 0.5 mL to 1.0 ML of the solution to a patient.

10. The method of claim 9 wherein the lung disease is COVID-19.

11. The method of claim 9 further comprising diluting tetrachlorodecaoxide to approximately 250 mL or more of 0.9% sodium chloride solution.

12. The system of claim 1 wherein the solution is contained within a nasal spray bottle and formulated to be delivered nasally.

13. The method of claim 9 wherein the solution is inhalable, further comprising storing the solution in a nasal spray bottle; and
aerosolizing the solution immediately prior to delivering the solution to a nasal cavity of a patient.

14. The method of claim 13 wherein a dose is provided to the patient twice daily for between three and five days.

15. The method of claim 13 wherein a dose of between 0.5 mL of 20% tetrachlorodecaoxide by volume and 1 mL of 30% tetrachlorodecaoxide.

16. The method of claim 9 wherein the solution is directly delivered to the nasal cavity or the rhinopharynx.

17. The method of claim 9 wherein the solution is aerosolized by an inhaler and inhaled by the patient.

18. The system of claim 1 wherein the solution is stored in an inhaler for aerosol delivery to the lungs of a patient.
